(19) United States
(12) Patent Application Publication
(10) Pub. No.: US 2016/0348998 A1
(43) Pub. Date: Dec. 1, 2016

(54) BOW HUNTING BOX

(71) Applicants: Virgil Larry Morrill, Minburn, IA (US); Ryan Lewis Ramsvig, Norwalk, IA (US)

(72) Inventors: Virgil Larry Morrill, Minburn, IA (US); Ryan Lewis Ramsvig, Norwalk, IA (US)

(21) Appl. No.: 15/170,735

(22) Filed: Jun. 1, 2016

Related U.S. Application Data
(60) Provisional application No. 62/169,365, filed on Jun. 1, 2015.

(51) Int. Cl. F41B 5/14; B65D 25/28; B65D 81/05; A61L 9/015; B65D 25/10; B65D 85/18; B62B 3/00; B01D 53/04; B65D 25/04; B65D 85/00

(52) U.S. Cl. CPC F41B 5/1457; B65D 25/04; B65D 25/28; B65D 81/05; B65D 85/70; B65D 25/102; B65D 85/187; B62B 3/004; B01D 53/04; A61L 9/015; B01D 2253/102; B01D 2257/90; B01D 2251/104

(57) ABSTRACT

The embodiments herein provide a bow hunting box for holding bow, hunting gear, hunting accessories, boots etc. The box comprises several compartments for holding bow tray, leg kit, ski kit etc. The bow tray holds the bow, arrows and hunting accessories in position using straps without damage. The box comprises an ozone device for maintaining the box scent free. The box comprises a lid for providing a dry platform for the hunter. The lid is configured to seal box with an O-ring gasket. The box is configured as a storage during off season for keeping the items away from pest, varmints, elements etc. The box is mounted on vehicle for easy transportation from one place to another. The box has carbon filter for removing the scent/odor.

### BACKGROUND

Technical Field

The embodiments herein are generally related to a multi-utility box for storage transport. The embodiments herein are particularly related to a multi-utility box for storing a plurality of hunting accessories in a single box. The embodiments herein are especially related a bow hunting box for storing all the hunting equipments and accessories in a single box during transport.

Description of the Related Art

Bow hunting is a hunting game enjoyed by many people. In bow hunting, a hunter has to wait in the wilderness for a long time for an encounter with an animal. Whenever there is an encounter with the animal, the hunter aims and fires an archery bow without frightening the animal. During the waiting period, the hunter needs to place the hunting accessories safely and in position so that the hunter can pick the accessories easily when the animal comes in the surrounding. Further, the hunter has to carry hunting bow, hunting accessories, boots, cloths, ski items etc, when the hunter move from place to place in the wilderness.

Various storage devices have been developed for storing the hunting accessories while moving the accessories to and from the hunting sites. In many cases, the hunter carries several additional items including boots, ski, clothes, knives, etc., along with the hunting accessories. However, carrying a lot of additional items along with the hunting accessories becomes tedious when the hunter moves to and from the hunting sites. Typically, the hunter tends to keep all the additional items in a separate container or in the storage means for storing hunting accessories.

However, storing the additional items separately in a bag makes it difficult for the hunter to move easily from one hunting site to the other. Further, there is always a chance of some items getting misplaced. Moreover, the storage of the additional items together in the storage device for hunting accessories tend to damage the hunting accessories. Further, there is a difficulty for the hunter in handling the accessories, when the additional items are kept loose in the storage means for storing the hunting accessories.

Hence, there is a need for a multi-utility box used as a bow hunting box for holding a plurality of hunting items including bow, hunting accessories, boots, clothes and the like. There is also a need for safely transporting the bow hunting box comprising the plurality of items safely from one place to another. Further, there is also a need for a convertible bow hunting box capable of storing the plurality of items including bow, hunting accessories, boots, clothes and the like, during off season without any damage. Still further there is a convertible bow hunting box capable of storing the plurality of hunting items in an air-tight, smell/scent/odor free and a waterproof/water tight structure.

The above mentioned shortcomings, disadvantages and problems are addressed herein and which will be understood by reading and studying the following specification.

### OBJECTS OF THE EMBODIMENTS

The primary object of the embodiments herein is to provide a bow hunting/sporting box for storing hunting/sporting accessories, goods, gear in an airtight, water proof and scent-free/proof box.

Another object of the embodiments herein is to provide a bow hunting box capable of holding and storing a plurality of items including bow, hunting gear, hunting accessories, hunter clothes and boots individually in separate compartments.

Yet another object of the embodiments herein is to provide a bow hunting box capable of being installed on a vehicle thereby permitting a transport of bow hunting box in a secure manner.

Yet another object of the embodiments herein is to provide a bow hunting box designed with an ozone device, for removing scent from the bow hunting box.

Yet another object of the embodiments herein is to provide a bow hunting box provided with a carbon filter that absorbs scents/smell/odor to make the items in the Bow Box scent free.

Yet another object of the embodiments herein is to provide a bow hunting box capable of being converted into a storage box during off season, thereby keeping the hunting accessories from pests, varmints, elements etc.

Yet another object of the embodiments herein is to provide a bow hunting box with a wheel, leg kit and a ski kit for enabling easier movement.

Yet another object of the embodiments herein is to provide a bow hunting box used for transportation in airplanes and vehicles to replace the multiple bags or totes for hunting/sporting gear or goods.

Yet another object of the embodiments herein is to provide a bow hunting box for storing and/or holding all your gear such as bow, hunting accessory, and clothing in one place.

Yet another object of the embodiments herein is to provide a bow hunting box with straps to hold and store the bow and the hunting accessories such as range finders, bow accessories in place and be strapped to keep from damage during travel and or storage.

Yet another object of the embodiments herein is to provide a bow hunting box made with plastics by injection mold processes to obtain a light weight box for carrying and transporting for bow and the hunting accessories while providing very good durability and design.

Yet another object of the embodiments herein is to provide a bow hunting box attached with easy to use latching handles.

Yet another object of the embodiments herein is to provide a bow hunting box with a plurality of wheels that stay recessed in the bottom of the box for mobility.

Yet another object of the embodiments herein is to provide a bow hunting box molded in the shape of the sledge for pulling through snow and ice.

Yet another object of the embodiments herein is to provide a bow hunting box sized to fit on an atv rack.

Yet another object of the embodiments herein is to provide a bow hunting box with ratchet straps or bungie straps to strap the plurality items down with.

Yet another object of the embodiments herein is to provide a bow hunting box with a ribbed lid is ribbed so that a strap is configured to slide from the top of the box and to keep the box in place during transport.

Yet another object of the embodiments herein is to provide a bow hunting box with holes for attaching the bungies to the box.

Yet another object of the embodiments herein is to provide a bow hunting box with holes to be used for paddle locks to secure items inside.

Yet another object of the embodiments herein is to provide a bow hunting box designed and sized to carry all hunting gear in one container, thereby eliminating a need for two containers.

Yet another object of the embodiments herein is to provide a bow hunting box sealed by an O-ring gasket to keep the box scent free.

Yet another object of the embodiments herein is to provide a bow hunting box with a Bow Tray having foam padding to protect the bow from damage.

Yet another object of the embodiments herein is to provide a bow hunting box with straps and dividers to keep the stored items separately and securely in place while transporting or storing.

Yet another object of the embodiments herein is to provide a bow hunting box that is converted to hold a cross bow.

Yet another object of the embodiments herein is to provide a bow hunting box that is designed to have dividers to convert the box into a fishing tackle/gear box, a camera/photography box, a livestock tack/and gear box.

Yet another object of the embodiments herein is to provide a bow hunting box with or built in with sledge design on a bottom of the box so that the box is used as an ice fishing sledge and gear box.

These and other objects and advantages of the embodiments herein will become readily apparent from the following detailed description taken in conjunction with the accompanying drawings.

### SUMMARY

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The various embodiments of the present invention provides a bow hunting box capable of holding bow, hunting gear, hunting accessories, hunting/hunter clothes, boots etc. The bow hunting box is a trapezoidal box having a plurality of compartments for holding bow tray, leg kit, ski kit etc. The bow tray holds the bow, arrows and hunting accessories in position. The leg kit comprises a plurality of items required to enable an easy movement of the bow hunting box from one hunting spot to another. Further, the ski kit comprises skiing equipments and accessories required for enabling a movement during winter season. The bow hunting box comprises an ozone device for maintaining the bow hunting box in a scent free condition. Further, the bow hunting box comprises a lid capable of providing a dry platform for the hunter. The lid also configured to seal the bow hunting box. The bow hunting bow is further used as a storage means during off season for keeping the plurality of stored items away from pest, varmints, elements etc. The bow hunting box is further capable of being mounted on vehicle for easy transportation from one place to another. The bow hunting box is provided with a carbon filter to remove the scent and to keep the box in a scent-free or odorless condition.

According to an embodiment herein, a bow hunting box is provided for storing hunting equipments and accessories. The bow hunting box comprises a plurality of storage compartments for storing a plurality of hunting equipments and accessories respectively. The plurality of storage compartments are formed by placing a plurality of divider panels in the bow hunting box. A pair of latching handles is attached to the opposite sides of the bow hunting box for lifting the bow hunting box. An ozone device is provided for removing bad scent from the bow hunting box. The ozone device acts as an air purifier. A lid for the box is provided with grip tapes at an inside surface for providing a dry platform for the hunter. The lid is placed on the ground upside down for providing the dry platform. A bow tray is placed in a first storage compartment for securely storing the plurality of hunting equipments. The bow tray comprises a plurality of straps for holding the plurality of hunting equipments. A wheel assembly is attached to the bottom surface of the box. The wheel assembly is recessed at the bottom of the bow hunting box for enabling mobility of the bow hunting box. The wheel assembly is engaged on lifting the bow hunting box. A sledge shaped design is provided at the bottom of the bow hunting box for enabling mobility during winter.

According to an embodiment herein, the plurality of hunting equipments and accessories are selected from a group consisting of bows, arrows, hunting clothes, boots, and range finders.

According to an embodiment herein, the bow tray further comprises a plurality of carbon filters for keeping the plurality of hunting equipments and hunting accessories free from pest, varmints, and elements.

According to an embodiment herein, the bow tray further comprises a foam padding for protecting the plurality of hunting equipments from damage during storage and transportation.

According to an embodiment herein, the bow tray is configured to hold the hunting equipments selected from a group consisting of bow, arrows, range finders.

According to an embodiment herein, the plurality of storage compartments further comprises a leg kit in a second storage compartment for storing hunting accessories including boots.

According to an embodiment herein, the plurality of storage compartments further comprises a ski kit in a third storage compartment for storing skiing accessories for the hunter.

According to an embodiment herein, the box lid is capable of securely sealing the bow hunting box using a plurality of latches.

According to an embodiment herein, the plurality of hunting equipments and accessories are stored securely during off season.

According to an embodiment herein, the plurality of hunting equipments and accessories are stored together in a single container without damage.

According to an embodiment herein, the bow hunting box is made of a plastic material using a mold injection process, and wherein the plastic is selected from a group consisting of polypropylene and high density polyethylene.

According to an embodiment herein, the bow hunting box is configured to be installed on vehicles using straps for secure transportation.

According to an embodiment herein, the bow hunting box further comprises a gasket for sealing the hunting box, and wherein the gasket is an O-ring gasket.

According to an embodiment herein, the bow hunting box further comprises a plurality of dividers for converting the hunting box into a fishing tackle/gear box, or a camera/photography box, or livestock/and gear box, or ice fishing/sled box.

According to an embodiment herein, the bow hunting box further comprises bungie straps or ratchet straps for fixing or fitting the hunting box with atv rack.

According to an embodiment herein, the lid is formed with ribs at outer surface.

According to an embodiment herein, a bow hunting box for storing the hunting equipments and accessories is disclosed. The bow hunting box comprises a plurality of storage compartments, a pair of latching handles, an ozone device, a lid, a bow tray, a wheel assembly and a sleigh. The plurality of storage compartments stores the hunting equipments, hunting gears, hunter/hunting clothes and hunting accessories. The plurality of storage compartments are formed by placing a plurality of divider panels in a tray in the bow hunting box. A pair of latching handles is attached to the opposite sides of the bow hunting bow for lifting the bow hunting box. The ozone device is configured to remove a bad scent/odor from the bow hunting box. The ozone device acts as an air purifier. The box lid comprises grip tapes at the inside surface for providing a dry platform for the hunter. The box lid is placed on the ground upside down for providing the dry platform. The bow tray placed in a first storage compartment for securely storing the hunting equipments. The bow tray comprises a plurality of straps for holding the hunting equipments. The wheel assembly is recessed at the bottom of the bow hunting box for enabling easy mobility of the bow hunting box. The wheel assembly is engaged on lifting the bow hunting box. The box is designed in a sledge shape for pulling the box through snow and winter. The sledge design is provided or built-in with sledge designed at the bottom of the bow hunting box for enabling an easy mobility during winter and snow.

According to an embodiment herein, the bow hunting box is configured to store the hunting equipments, the hunting gears and accessories selected from a group consisting of bows, arrows, hunting clothes, boots, range finders and the like.

According to an embodiment herein, the bow tray comprises a plurality of carbon filters for keeping the hunting equipments and accessories free from pest, varmints, elements etc.

According to an embodiment herein, the bow tray further comprises foam padding for protecting the hunting equipments from damage.

According to an embodiment herein, the bow tray holds the hunting equipments selected from a group consisting of bow, arrows, range finders etc.

According to an embodiment herein, the plurality of storage compartments further comprises a second storage compartment comprising a leg kit for storing the hunting accessories including boots.

According to an embodiment herein, the plurality of storage compartments further comprises a third storage compartment comprising a ski kit for storing the skiing accessories for the hunter.

According to an embodiment herein, the lid is capable of securely sealing the bow hunting box using a plurality of latches.

According to an embodiment herein, the bow hunting box is capable of storing the hunting equipments and hunting accessories securely during off season.

According to an embodiment herein, the bow hunting box is capable of holding the hunting equipments and accessories together in a single container without damage.

According to an embodiment herein, the bow hunting box is made with a plastic material using an injection molding process. The plastic material is selected from a group consisting of polypropylene and high density polyethylene etc.

According to an embodiment herein, the bow hunting box is capable of being installed on the vehicles using straps for secure transportation.

According to an embodiment herein, the bow hunting/sporting box is configured for storing hunting/sporting accessories, goods, gear in an airtight, water proof and scent-free/proof box.

According to an embodiment herein, the bow hunting box is configured for holding and storing a plurality of items including bow, hunting gear, hunting accessories, hunter clothes and boots individually in separate compartments.

According to an embodiment herein, the bow hunting box is configured to be installed on a vehicle thereby permitting a transport of bow hunting box in a secure manner.

According to an embodiment herein, the bow hunting box is designed to have an ozone device, for removing scent from the bow hunting box.

According to an embodiment herein, the bow hunting box is provided with a carbon filter that absorbs scents/smell/odor to make the items in the Bow Box scent free.

According to an embodiment herein, the bow hunting box is configured to be converted into a storage box during off season, thereby keeping the hunting accessories from pests, varmints, elements etc.

According to an embodiment herein, the bow hunting box is provided with a wheel, leg kit and a ski kit for enabling easier movement.

According to an embodiment herein, the bow hunting box is configured for transportation in airplanes and vehicles to replace the multiple bags or totes for hunting/sporting gear or goods.

According to an embodiment herein, the bow hunting box is configured for storing and/or holding all your gear such as bow, hunting accessory, and clothing in one place.

According to an embodiment herein, the bow hunting box is provided with straps to hold and store the bow and the hunting accessories such as range finders, bow accessories in place and be strapped to keep from damage during travel and or storage.

According to an embodiment herein, the bow hunting box is made of plastics using an injection mold processes to obtain a light weight box for carrying and transporting for bow and the hunting accessories while providing very good durability and design.

According to an embodiment herein, the bow hunting box is attached with easy to use latching handles.

According to an embodiment herein, the bow hunting box is provided with a plurality of wheels that stay recessed in the bottom of the box for mobility.

According to an embodiment herein, the bow hunting box is molded in the shape of the sledge for pulling through snow and ice.

According to an embodiment herein, the bow hunting box is sized to fit on an atv rack.

According to an embodiment herein, the bow hunting box is provided with ratchet straps or bungie straps to strap the plurality items down with.

According to an embodiment herein, the bow hunting box is provided with a ribbed lid is ribbed so that a strap is configured to slide from the top of the box and to keep the box in place during transport.

According to an embodiment herein, the bow hunting box is provided with holes for attaching the bungies to the box.

According to an embodiment herein, the bow hunting box is provided with holes to be used for paddle locks to secure items inside.

According to an embodiment herein, the bow hunting box is designed and sized to carry all hunting gear in one container, thereby eliminating a need for two containers.

According to an embodiment herein, the bow hunting box is sealed by an O-ring gasket to keep the box scent free.

According to an embodiment herein, the bow hunting box is provided with a bow tray having foam padding to protect the bow from damage.

According to an embodiment herein, the bow hunting box is provided with straps and dividers to keep the stored items separately and securely in place while transporting or storing.

According to an embodiment herein, the bow hunting box is converted to hold a cross bow.

According to an embodiment herein, the bow hunting box that is designed to have dividers to convert the box into a fishing tackle/gear box, a camera/photography box, a livestock tack/and gear box.

According to an embodiment herein, the bow hunting box is designed with or built in with sledge design on a bottom of the box so that the box is used as an ice fishing sledge and gear box.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### BRIEF DESCRIPTION OF THE DRAWINGS

The other objects, features and advantages will occur to those skilled in the art from the following description of the preferred embodiment and the accompanying drawings in which:

FIG. 1A illustrates a front side view of a bow hunting box, according to one embodiment herein.

FIG. 1B illustrates a side view of a bow hunting box, according to one embodiment herein.

FIG. 1C illustrates a top side view of a lid of the bow hunting box, according to one embodiment herein.

FIG. 1D illustrates a bottom side view of a lid of the bow hunting box, according to one embodiment herein.

FIG. 1E illustrates a top side view of a bow hunting box in a lid removed condition, according to one embodiment herein.

FIG. 2A illustrates a top side view of a bow tray in a bow hunting box in a lid removed condition, according to one embodiment herein.

FIG. 2B illustrates a front side view of a bow tray in a bow hunting box, according to one embodiment herein.

FIG. 2C illustrates a side view of a bow tray in a bow hunting box, according to one embodiment herein.

FIG. 2D illustrates a bottom side view of a bow tray in a bow hunting box, according to one embodiment herein.

FIG. 3 illustrates a front side view of a leg kit in a bow hunting box, according to one embodiment herein.

FIG. 4A illustrates a front side view of a ski kit in a bow hunting box, according to one embodiment herein.

FIG. 4B illustrates a rear side view of a ski kit in a bow hunting box, according to one embodiment herein.

FIG. 5 illustrates a flow chart explaining a method of storing the plurality of items in the bow hunting box at a hunting spot, according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments of the present invention provides a bow hunting box capable of holding bow, hunting gear, hunting accessories, hunting/hunter clothes, boots etc. The bow hunting box is a trapezoidal box having a plurality of compartments for holding bow tray, leg kit, ski kit etc. The bow tray holds the bow, arrows and hunting accessories in position. The leg kit comprises a plurality of items required to enable an easy movement of the bow hunting box from one hunting spot to another. Further, the ski kit comprises skiing equipments and accessories required for enabling a movement during winter season. The bow hunting box comprises an ozone device for maintaining the bow hunting box in a scent free condition. Further, the bow hunting box comprises a lid capable of providing a dry platform for the hunter. The lid also configured to seal the bow hunting box. The bow hunting bow is further used as a storage means during off season for keeping the plurality of stored items away from pest, varmints, elements etc. The bow hunting box is further capable of being mounted on vehicle for easy transportation from one place to another the bow hunting box is provided with a carbon filter to remove the scent and to keep the box in a scent-free or odorless condition.

According to an embodiment herein, the plurality of hunting equipments and accessories are selected from a group consisting of bows, arrows, hunting clothes, boots, and range finders.

According to an embodiment herein, the bow tray further comprises a plurality of carbon filters for keeping the plurality of hunting equipments and hunting accessories free from pest, varmints, and elements.

According to an embodiment herein, the bow tray further comprises a foam padding for protecting the plurality of hunting equipments from damage during storage and transportation.

According to an embodiment herein, the bow tray is configured to hold the hunting equipments selected from a group consisting of bow, arrows, and range finders.

According to an embodiment herein, the plurality of storage compartments further comprises a leg kit in a second storage compartment for storing hunting accessories including boots.

According to an embodiment herein, the plurality of storage compartments further comprises a ski kit in a third storage compartment for storing skiing accessories for the hunter.

According to an embodiment herein, the box lid is capable of securely sealing the bow hunting box using a plurality of latches.

According to an embodiment herein, the plurality of hunting equipments and accessories are stored securely during off season.

According to an embodiment herein, the plurality of hunting equipments and accessories are stored together in a single container without damage.

According to an embodiment herein, the bow hunting box is made of a plastic material using a mold injection process, and wherein the plastic is selected from a group consisting of polypropylene and high density polyethylene.

According to an embodiment herein, the bow hunting box is configured to be installed on vehicles using straps for secure transportation.

According to an embodiment herein, the bow hunting box further comprises a gasket for sealing the hunting box, and wherein the gasket is an O-ring gasket.

According to an embodiment herein, the bow hunting box further comprises a plurality of dividers for converting the hunting box into a fishing tackle/gear box, or a camera/photography box, or livestock/and gear box, or ice fishing/sled box.

According to an embodiment herein, the bow hunting box further comprises bungie straps or ratchet straps for fixing or fitting the hunting box with atv rack.

According to an embodiment herein, the lid is formed with ribs at outer surface.

According to an embodiment herein, a bow hunting box for storing the hunting equipments and accessories is disclosed. The bow hunting box comprises a plurality of storage compartments, a pair of latching handles, an ozone device, a lid, a bow tray, a wheel assembly and a sleigh. The plurality of storage compartments stores the hunting equipments, hunting gears, hunter/hunting clothes and hunting accessories. The plurality of storage compartments are formed by placing a plurality of divider panels in a tray in the bow hunting box. A pair of latching handles is attached to the opposite sides of the bow hunting bow for lifting the bow hunting box. The ozone device is configured to remove a bad scent/odor from the bow hunting box. The ozone device acts as an air purifier. The box lid comprises grip tapes at the inside surface for providing a dry platform for the hunter. The box lid is placed on the ground upside down for providing the dry platform. The bow tray placed in a first storage compartment for securely storing the hunting equipments. The bow tray comprises a plurality of straps for holding the hunting equipments. The wheel assembly is recessed at the bottom of the bow hunting box for enabling easy mobility of the bow hunting box. The wheel assembly is engaged on lifting the bow hunting box. The box is designed in a sledge shape for pulling the box through snow and winter. The sledge design is provided or built-in with sledge designed at the bottom of the bow hunting box for enabling an easy mobility during winter and snow.

According to an embodiment herein, the bow hunting box is configured to store the hunting equipments, the hunting gears and accessories selected from a group consisting of bows, arrows, hunting clothes, boots, range finders and the like.

According to an embodiment herein, the bow tray comprises a plurality of carbon filters for keeping the hunting equipments and accessories free from pest, varmints, elements etc.

According to an embodiment herein, the bow tray further comprises foam padding for protecting the hunting equipments from damage.

According to an embodiment herein, the bow tray holds the hunting equipments selected from a group consisting of bow, arrows, range finders etc.

According to an embodiment herein, the plurality of storage compartments further comprises a second storage compartment comprising a leg kit for storing the hunting accessories including boots.

According to an embodiment herein, the plurality of storage compartments further comprises a third storage compartment comprising a ski kit for storing the skiing accessories for the hunter.

According to an embodiment herein, the lid is capable of securely sealing the bow hunting box using a plurality of latches.

According to an embodiment herein, the bow hunting box is capable of storing the hunting equipments and hunting accessories securely during off season.

According to an embodiment herein, the bow hunting box is capable of holding the hunting equipments and accessories together in a single container without damage.

According to an embodiment herein, the bow hunting box is made with a plastic material using an injection molding process. The plastic material is selected from a group consisting of polypropylene and high density polyethylene etc.

According to an embodiment herein, the bow hunting box is capable of being installed on the vehicles using straps for secure transportation.

According to an embodiment herein, the bow hunting/sporting box is configured for storing hunting/sporting accessories, goods, gear in an airtight, water proof and scent-free/proof box.

According to an embodiment herein, the bow hunting box is configured for holding and storing a plurality of items including bow, hunting gear, hunting accessories, hunter clothes and boots individually in separate compartments.

According to an embodiment herein, the bow hunting box is configured to be installed on a vehicle thereby permitting a transport of bow hunting box in a secure manner.

According to an embodiment herein, the bow hunting box is designed to have an ozone device, for removing scent from the bow hunting box.

According to an embodiment herein, the bow hunting box is provided with a carbon filter that absorbs scents/smell/odor to make the items in the Bow Box scent free.

According to an embodiment herein, the bow hunting box is configured to be converted into a storage box during off season, thereby keeping the hunting accessories from pests, varmints, elements etc.

According to an embodiment herein, the bow hunting box is provided with a wheel, leg kit and a ski kit for enabling easier movement.

According to an embodiment herein, the bow hunting box is configured for transportation in airplanes and vehicles to replace the multiple bags or totes for hunting/sporting gear or goods.

According to an embodiment herein, the bow hunting box is configured for storing and/or holding all your gear such as bow, hunting accessory, and clothing in one place.

According to an embodiment herein, the bow hunting box is provided with straps to hold and store the bow and the hunting accessories such as range finders, bow accessories in place and be strapped to keep from damage during travel and or storage.

According to an embodiment herein, the bow hunting box is made of plastics using an injection mold processes to obtain a light weight box for carrying and transporting for bow and the hunting accessories while providing very good durability and design.

According to an embodiment herein, the bow hunting box is attached with easy to use latching handles.

According to an embodiment herein, the bow hunting box is provided with a plurality of wheels that stay recessed in the bottom of the box for mobility.

According to an embodiment herein, the bow hunting box is molded in the shape of the sledge for pulling through snow and ice.

According to an embodiment herein, the bow hunting box is sized to fit on an atv rack.

According to an embodiment herein, the bow hunting box is provided with ratchet straps or bungie straps to strap the plurality items down with.

According to an embodiment herein, the bow hunting box is provided with a ribbed lid is ribbed so that a strap is configured to slide from the top of the box and to keep the box in place during transport.

According to an embodiment herein, the bow hunting box is provided with holes for attaching the bungies to the box.

According to an embodiment herein, the bow hunting box is provided with holes to be used for paddle locks to secure items inside.

According to an embodiment herein, the bow hunting box is designed and sized to carry all hunting gear in one container, thereby eliminating a need for two containers.

According to an embodiment herein, the bow hunting box is sealed by an O-ring gasket to keep the box scent free.

According to an embodiment herein, the bow hunting box is provided with a bow tray having foam padding to protect the bow from damage.

According to an embodiment herein, the bow hunting box is provided with straps and dividers to keep the stored items separately and securely in place while transporting or storing.

According to an embodiment herein, the bow hunting box is converted to hold a cross bow.

According to an embodiment herein, the bow hunting box that is designed to have dividers to convert the box into a fishing tackle/gear box, a camera/photography box, a livestock tack/and gear box.

According to an embodiment herein, the bow hunting box is designed with or built in with sledge design on a bottom of the box so that the box is used as an ice fishing sledge and gear box.

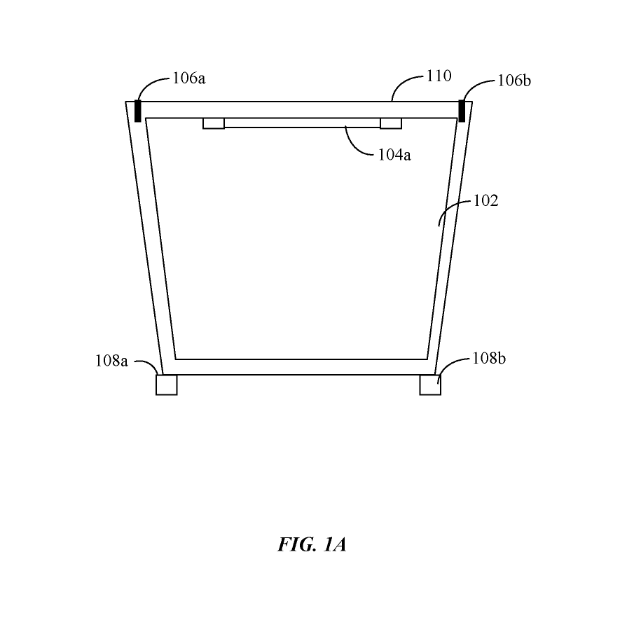

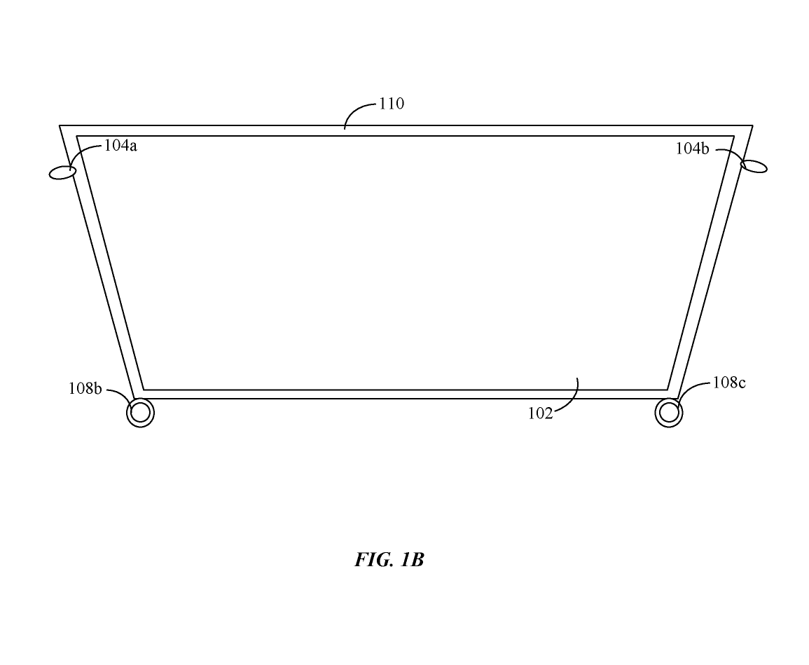

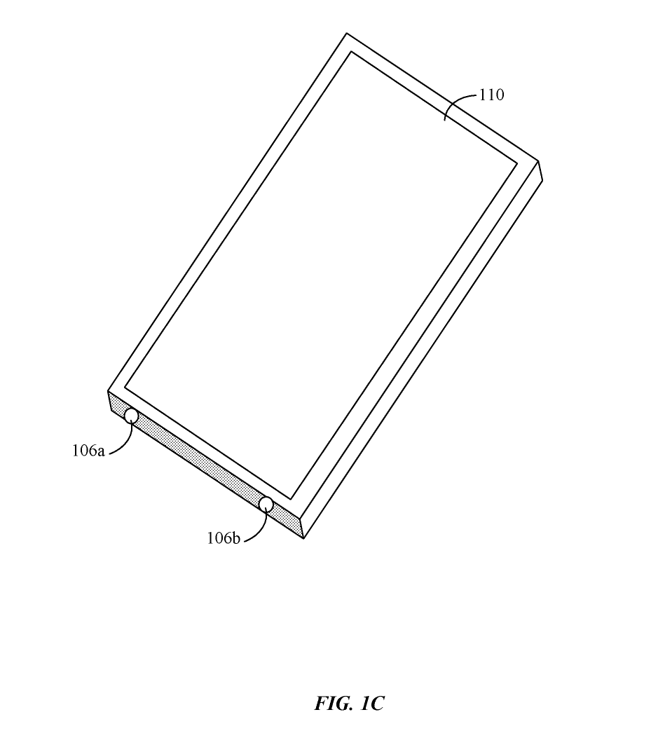

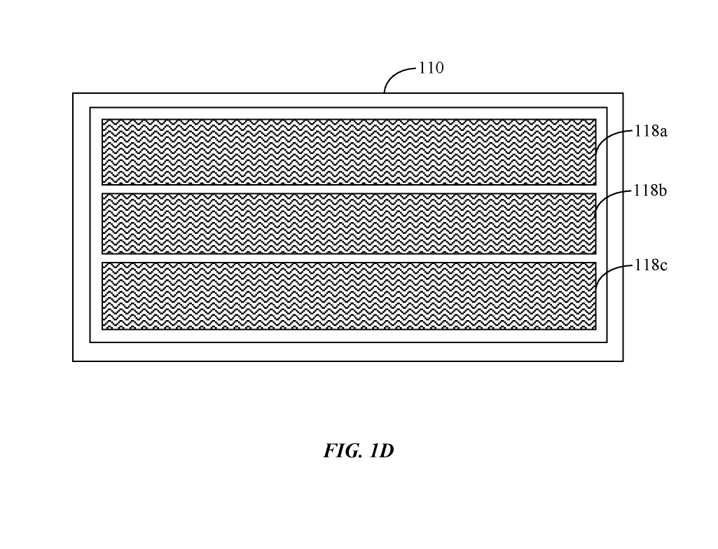

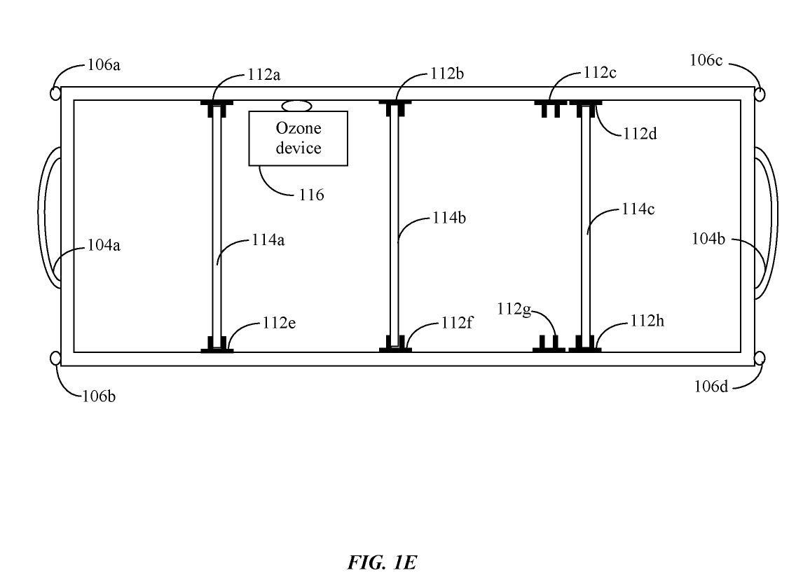

FIG. 1A illustrates a front side view of a bow hunting box, according to one embodiment herein. FIG. 1B illustrates a side view of a bow hunting box, according to one embodiment herein. FIG. 1C illustrates a top side view of a lid of the bow hunting box, according to one embodiment herein. FIG. 1D illustrates a bottom side view of a lid of the bow hunting box, according to one embodiment herein. FIG. 1E illustrates a top side view of a bow hunting box in a lid removed condition, according to one embodiment herein.

With respect to FIG. 1A-FIG. 1B, the bow hunting box 102 is designed to hold a plurality of items including but not limited to bow, bow accessories, hunting gears, range finders, hunting clothes etc. The bow hunting box 102 is a trapezoidal shape box comprising a plurality of compartments for holding the plurality of items. The bow hunting box 102 comprises a bow tray, a leg kit and ski kit for holding the plurality of items in the plurality of compartments. The bow hunting box 102 comprises a pair of handles 104a and 104b, a box lid 110, a plurality of divider panels 114a, 114c and 114d, and an ozone device 116.

FIG. 1A-FIG. 1E illustrates the external appearance of the bow hunting box 102. The pair of handles 104a and 104b are attached to the opposite sides of the bow hunting box 102. The pair of handles 104a and 104b comprises a first handle 104a attached to a front side of the bow hunting box 102 and a second handle 104b attached to the rear side of the bow hunting box 102. The pair of handles 104a and 104b are configured to easily lift and handle the bow hunting box 102. The bow hunting box 102 further comprises a plurality of wheels 108a, 108b, 108c and 108d attached to the bottom surface of the box and recessed at the bottom of the bow hunting box 102. The plurality of wheels 108a, 108b, 108c and 108d is engaged by lifting up the bow hunting box 102. The plurality of wheels 108a, 108b, 108c and 108d enables easy movement of the bow hunting box 102 from one hunting site to the other. Further, the bottom portion of the bow hunting box 102 is designed in the model of a sledge design/shape thereby enabling easy transportation of the bow hunting box 102 from one hunting site to the other during winter season.

The bow hunting box 102 further comprises a box lid 110 for proper sealing. The box lid 110 enables a proper sealing of the items in the bow hunting box 102. The top view and bottom view of the box lid 110 are illustrated in FIG. 1C and FIG. 1D. The box lid 110 comprises a plurality of latches 106a, 106b 106c and 106d provided respectively at the four corners for tightly fastening the box lid 110 with the bow hunting box 102. The plurality of latches 106a, 106b 106c and 106d are flexible latches and comprises a hook extending to hold the box lid 110 properly. Once the box lid 110 is placed on top of the bow hunting box, the plurality of latches 106a, 106b 106c and 106d are selectively placed in position. The bottom side of the lid box 110 comprises a plurality of grip tapes 118a, 118b and 118c. The lid box 110 is laid on the ground to provide a dry platform for the hunter. The box lid 110 is laid on the ground upside down with plurality of grip tapes 118a, 118b and 118c facing upwards. Thus, the hunter is enabled to step onto the box the lid 110 for changing into hunting clothes, thereby avoiding the need of stepping onto cold or wet ground. The hunter enabled to stand firmly at the hunting spot without the fear of slipping or falling down.

Further, FIG. 1E illustrates the inside of the bow hunting box 102. The bow hunting box 102 comprises a plurality of dividers 112a, 112b . . . 112h for holding the plurality of divider panels 114a, 114c and 114d. The plurality of dividers 112a, 112b . . . 112h is made of 6 mm thick plastic material. The plurality of divider panels 114a, 114c and 114d are made of 6 mm thick PVC or cutting board material. Each divider panel is inserted on a slot formed by two dividers placed on either side of the bow hunting box. Each divider panel among the plurality of divider panels 114a, 114c and 114d are inserted on different slots thereby making a plurality of compartments in the bow hunting box 102. The divider panels are inserted depending on the requirement of width of each compartment. The plurality of compartments stores different items including the bow tray, the leg kit, ski kit etc. The divider panels are available in different colors including orange, gray and pink.

Further, the ozone device 116 is attached at the inside of the bow hunting box 102. The ozone device 116 is an air purifier. The ozone device purifies the air in the bow hunting box 102, thereby keeping the bow hunting box 102 in a scent free condition. Thus the plurality of items stored in the bow hunting box such as boots and clothes are also maintained scent free. The hunter on reaching a hunting site is enabled to open the box lid 110 and wear clothes that are scent free from the bow hunting box 102. Further, the ozone device 116 is also configured to maintain/hold/store the items inside the bow hunting box 102 in a scent free condition, while storing the items in the off season.

The bow hunting box 102 is further capable of being installed at any one of a a truck, four wheeler racks, or vehicle rear car carrier rack. The bow hunting box 102 is strapped down with ratchet straps or bungie straps on the vehicles. The box lid 110 is designed to have ribbed surface to ensure that the strap are designed from sliding from the top of the box and keep the box in place during transport. The bow hunting box is securely attached to a vehicle thereby enabling easy and convenient transfer from one place to another. Further, the bow hunting box 102 is made of plastics using an injection mold process. The plastic material is selected from a group consisting of polypropylene and high density polyethylene etc. Therefore, the bow hunting box is lightweight and durable.

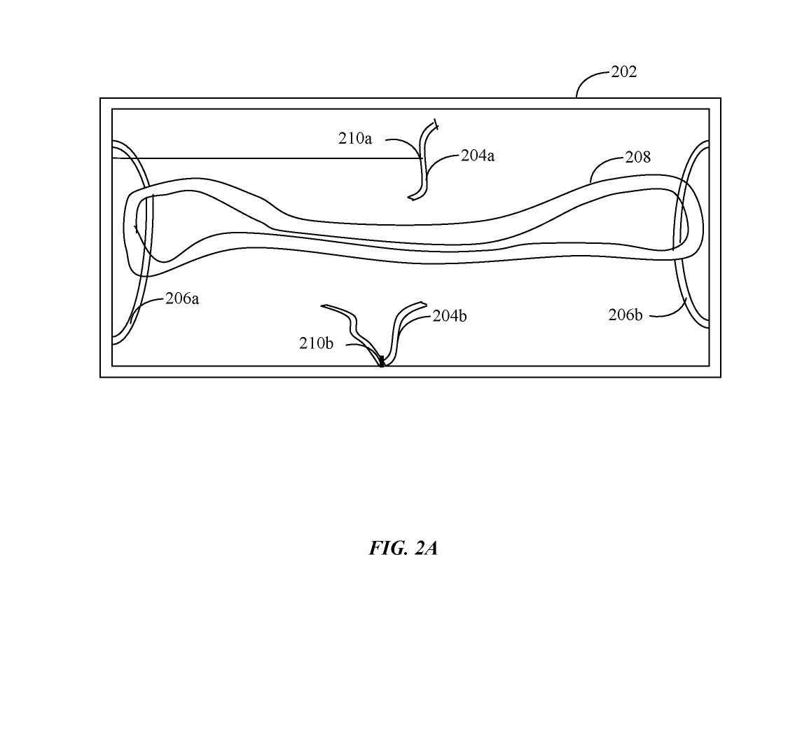

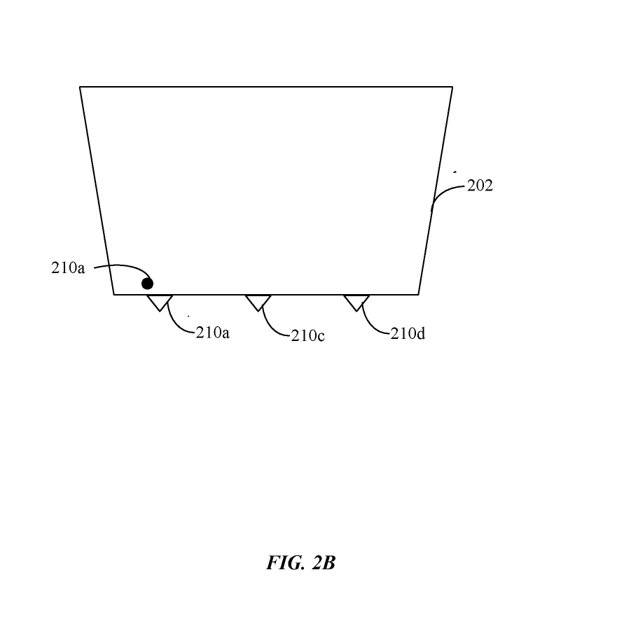

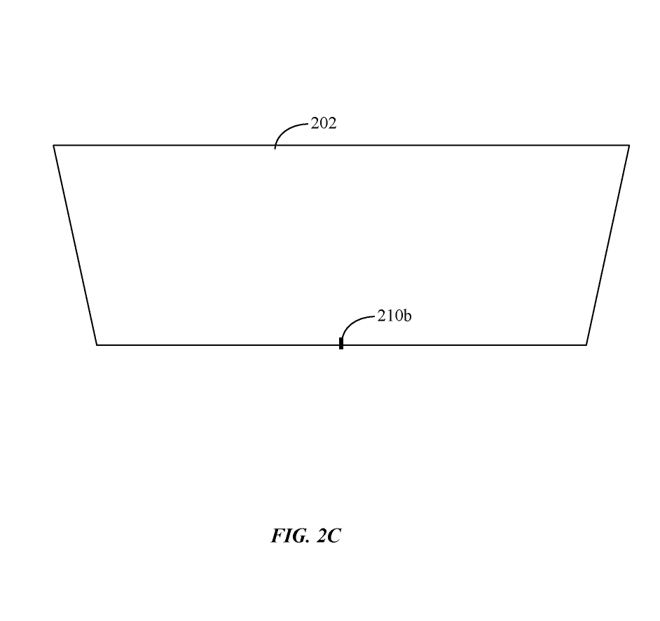

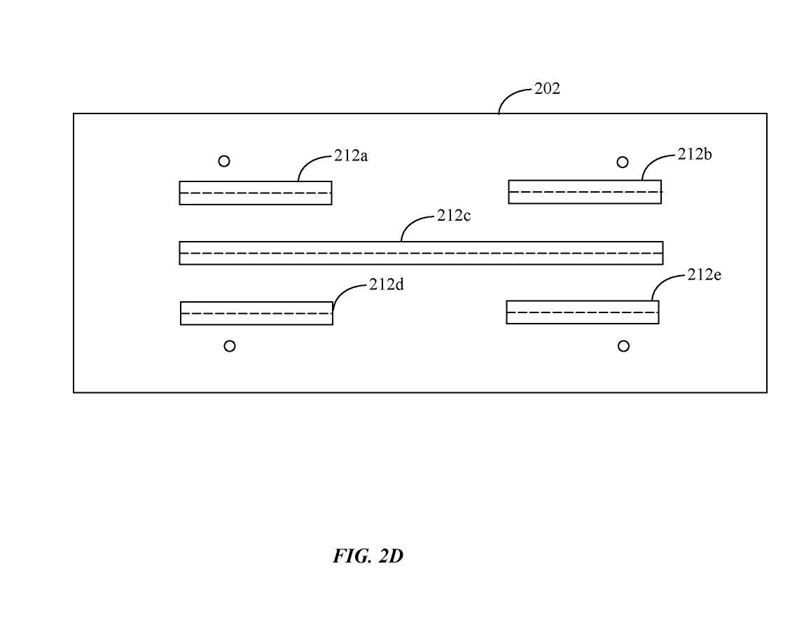

FIG. 2A illustrates a top side view of a bow tray in a bow hunting box in a lid removed condition, according to one embodiment herein. FIG. 2B illustrates a front side view of a bow tray in a bow hunting box, according to one embodiment herein. FIG. 2C illustrates a side view of a bow tray in a bow hunting box, according to one embodiment herein. FIG. 2D illustrates a bottom side view of a bow tray in a bow hunting box, according to one embodiment herein.

With respect to FIG. 2A-FIG. 2D, the bow tray 202 is arranged inside the bow hunting box 102 in a trapezoidal shape. The bow tray 202 is fit into a first compartment among the plurality of compartments in the bow hunting box 102. The bow tray 202 is designed to hold the hunting equipments including a bow, a bunch of arrows, a range finder etc., in position. The bow tray 202 is provided with straps to fasten the hunting equipments, thereby avoiding the damage of hunting equipments during storage and transportation. The bow tray is provided with a pad made of soft foam material for storing the items without any damage. The tray is provided with a plurality of dividers for forming a plurality of compartments for securing and storing the plurality of items.

The bow tray 202 comprises a pair of velcro straps 204a and 204b, a pair of nylon straps 206a and 206b, an adjustable strap 208 and a pair of rivets 210a and 210b, a plurality of carbon filters 212a, 212b . . . 212e. The pair of velcro straps 204a and 204b are riveted using pair of rivets 210a and 210b. The first velcro strap 204a is riveted at the bottom of the bow tray using a first rivet 210a. The second velcro strap 204b is riveted using the second rivet 210b. The pair of velcro straps 204a and 204b is fastened to secure a bunch of arrows. The rivet 210 is fixed at a center point at the bottom of the bow tray 202.

Further, the pair of nylon straps 206a and 206b is attached to the two opposite sides of the bow tray 202. The pair of nylon straps is designed to hold the adjustable strap 208. The adjustable strap 208 comprises a buckle for adjusting the length. The adjustable strap 208 is made of a six inch long nylon material. The adjustable strap 204 is designed to hold the bow tightly by adjusting the length by pulling the strap through the buckle.

Further, the plurality of carbon filters 212a, 212b . . . 212e is placed at the bottom of the bow tray 202. The plurality of carbon filters 212a, 212b . . . 212e are used as air filtering system that suck and remove bad air. The plurality of carbon filters 212a, 212b 212e comprises activated carbon granules for absorbing organic contaminants from the air inside the bow tray. The plurality of carbon filters 212a, 212b . . . 212e is used to keep the items in the bow tray and bow hunting box free from pest, varmints, elements etc while using the bow hunting box as storage during off season. The plurality of carbon filters 212a, 212b . . . 212e are attached to the bottom of the bow tray 202 using straps. The hunter on reaching a hunting spot is enabled to take the bow tray 202 out and keep aside for picking up the required equipments. The plurality of carbon filters 212a, 212b . . . 212e filters the air and keeps the hunting equipments in the bow tray scent free.

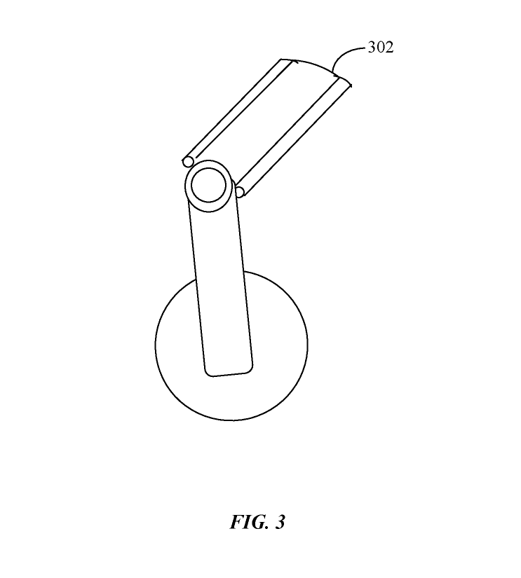

FIG. 3 illustrates a front side view of a leg kit in a bow hunting box, according to one embodiment herein. The leg kit 302 comprises accessories for enabling easy movement from one hunting place to another. The leg kit 302 comprises a pair of wheels, boots and four axle pins. The leg kit 302 is stored in a second compartment among the plurality of compartments in the bow hunting box.

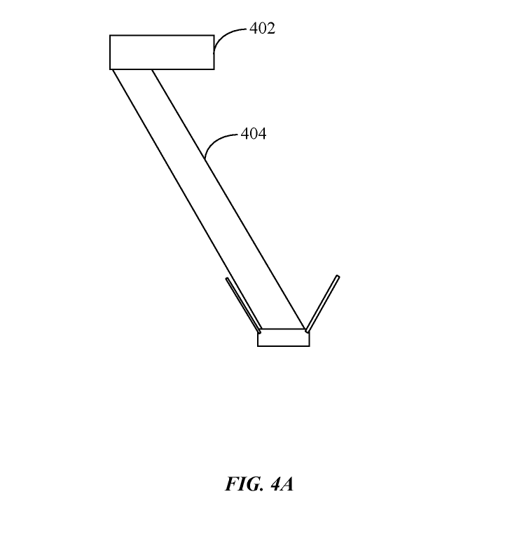

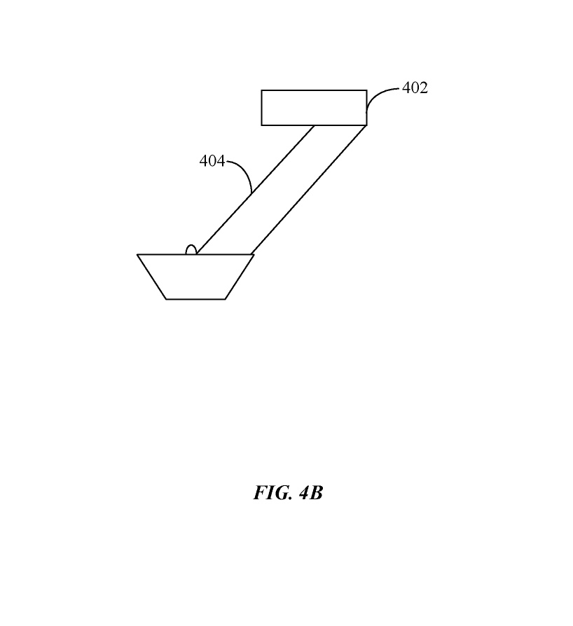

FIG. 4A illustrates a front side view of a ski kit in a bow hunting box, according to one embodiment herein. FIG. 4B illustrates a rear side view of a ski kit in a bow hunting box, according to one embodiment herein. The ski kit comprises a bushing 402 and an angle 404. The ski kit is designed to hold skiing equipment and accessories for enabling movement during the winter season.

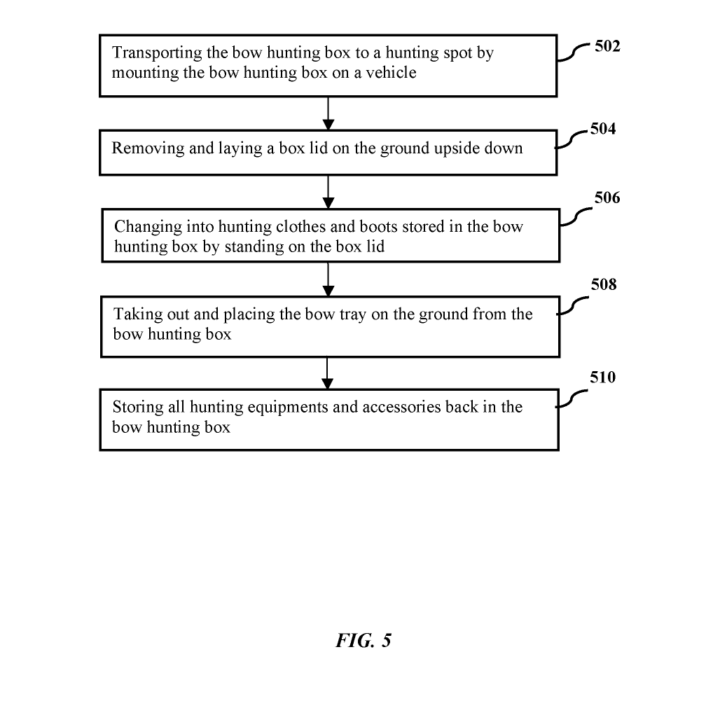

FIG. 5 illustrates a flow chart explaining a method of using the bow hunting box at a hunting spot, according to one embodiment herein. The bow hunting box is transported to the hunting spot by mounting the bow hunting box on a vehicle (502). The bow hunting box is capable of being installed at the back of a truck, four wheeler racks, or back of vehicle rear car carrier rack. The bow hunting box is strapped down with straps on the vehicles. Further, a box lid is ribbed to ensure that the strap keeps from sliding from the top of the box and keep the box in place during transport.

Once the hunting spot is reached the hunter removes and lays the box lid on the ground upside down (504). The inner side of the box lid comprises grip tapes, thereby providing a dry platform for the hunter. The hunter is further enabled to change into hunting clothes and boots stored in the bow hunting box by standing on the box lid (506). Further, the hunter is enabled to take out and place the bow tray on the ground from the bow hunting box (508). The hunter is enabled to reach out for different hunting equipments and accessories to the bow tray. Once the hunting is over, the hunter is enabled to store all hunting equipments and accessories back in the bow hunting box, thereby protecting the hunting equipments and accessories from pests, varmints, elements, bad odor etc (510).

Therefore, bow hunting box is capable of holding all hunting equipments and accessories in a single box, thereby avoiding the use of multiple bags. The use of a single bow hunting box is highly advantageous while transporting the hunting equipments and accessories especially for large distance. The bow hunting box is useful in transporting the hunting equipments and accessories even in airplanes. The bow hunting box is also capable of being mounted on vehicles for easy transportation from one hunting site to another. Further, the bow hunting box is lightweight and durable which ease the transportation.

Further, the bow hunting box is capable of maintaining all the hunting equipments and accessories scent free. The bow hunting box is further useful in storing hunting equipments and accessories safely and securely during off season. The bow hunting box is an airtight, waterproof and scent proof, thereby protecting the hunting equipments and accessories from pest, varmints, elements, bad odor etc.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A bow hunting box for storing hunting equipments and accessories, wherein the bow hunting box comprising:
a plurality of storage compartments for storing a plurality of hunting equipments and accessories respectively, wherein the plurality of storage compartments are formed by placing a plurality of divider panels in the bow hunting box;
a pair of latching handles attached to opposite sides of the bow hunting box for lifting the bow hunting box;
an ozone device for removing bad scent from the bow hunting box wherein the ozone device acts as an air purifier;
a box lid with grip tapes at an inside surface for providing a dry platform for the hunter, wherein the box lid is placed on the ground upside down for providing the dry platform;
a bow tray placed in a first storage compartment for securely storing the plurality of hunting equipments, wherein the bow tray comprises a plurality of straps for holding the plurality of hunting equipments;
a wheel assembly recessed at the bottom of the bow hunting box for enabling mobility of the bow hunting box, wherein the wheel assembly is engaged on lifting the bow hunting box; and
a sledge shaped design provided at the bottom of the bow hunting box for enabling mobility during winter.

2. The bow hunting box according to claim 1, wherein the plurality of hunting equipments and accessories are selected from a group consisting of bows, arrows, hunting clothes, boots, and range finders.

3. The bow hunting box according to claim 1, wherein the bow tray further comprises a plurality of carbon filters for keeping the plurality of hunting equipments and hunting accessories free from pest, varmints, and elements.

4. The bow hunting box according to claim 1, wherein the bow tray further comprises a foam padding for protecting the plurality of hunting equipments from damage during storage and transportation.

5. The bow hunting box according to claim 1, wherein the bow tray is configured to hold the hunting equipments selected from a group consisting of bow, arrows, range finders.

6. The bow hunting box according to claim 1, wherein the plurality of storage compartments further comprises a leg kit in a second storage compartment for storing hunting accessories including boots.

7. The bow hunting box according to claim 1, wherein the plurality of storage compartments further comprises a ski kit in a third storage compartment for storing skiing accessories for the hunter.

8. The bow hunting box according to claim 1, wherein the box lid is capable of securely sealing the bow hunting box using a plurality of latches.

9. The bow hunting box according to claim 1, wherein the plurality of hunting equipments and accessories are stored securely during off season.

10. The bow hunting box according to claim 1, wherein the plurality of hunting equipments and accessories are stored together in a single container without damage.

11. The bow hunting box according to claim 1, wherein the bow hunting box is made of a plastic material using a mold injection process, and wherein the plastic is selected from a group consisting of polypropylene and high density polyethylene.

12. The bow hunting box according to claim 1, wherein the bow hunting box is configured to be installed on vehicles using straps for secure transportation.

13. The bow hunting box according to claim 1, further comprises a gasket for sealing the hunting box, and wherein the gasket is an O-ring gasket.

14. The bow hunting box according to claim 1, further comprises a plurality of dividers for converting the hunting box into a fishing tackle/gear box, or a camera/photography box, or livestock/and gear box, or ice fishing/sled box.

15. The bow hunting box according to claim 1, further comprises bungie straps or ratchet straps for fixing or fitting the hunting box with atv rack.

16. The bow hunting box according to claim 1, wherein the lid is formed with ribs at outer surface.
